(19) United States
(12) Patent Application Publication
(10) Pub. No.: US 2003/0163041 A1
(43) Pub. Date: Aug. 28, 2003

(54) Catheter with lumen for contrast material fluid

(76) Inventor: William N. Paik, West Covina, CA (US)

Correspondence Address: CHARLES C.H. WU, 7700 IRVINE CENTER DRIVE, SUITE 710, IRVINE, CA, 92618-3043, US

(21) Appl. No.: 10/079,750

(22) Filed: Feb. 22, 2002

(51) Int. Cl. A61B 6/00

(52) U.S. Cl. 600/431

(57) ABSTRACT

A catheter chat allows to introduce contrast material fluid into the blood vessel for visualizing vascular anatomy and shape without the need to retract a guide wire is disclosed. In the first embodiment of this invention, a single lumen is used for both guide wire insertion and as a path for contrast material fluid introduced through a conduit tube which is in fluid communication with the lumen. In the second embodiment of this invention, a first lumen is used for guide wire insertion, whereas a second lumen is used as a path for contrast material fluid introduced through a conduit tube which is in fluid communication with the second lumen. In both these embodiments, contrast material fluid can be introduced into the blood vessel without the need to retract the guide wire.

### FIELD OF THE INVENTION

The present invention relates to a catheter with lumen for contrast material fluid, and more specifically, a catheter that allows to introduce contrast material fluid without the need to retract a guide wire.

### BACKGROUND OF THE INVENTION

Catheters are widely used in endovascular procedures, both for the purposes of diagnoses and intervention. A catheter is substantially an elongated tube housing a flexible guide wire that can slide within the catheter. The guide wire is inserted into a lumen of a blood vessel and then pushed, allowing it to “find its way” through the complex anatomy of the vessel.

It is often necessary to visualize the anatomy and shape of the vessel. To do this, contrast material fluid is injected into the vessel. An iodine contained in the contrast material fluid is visible on an X-ray, which allows the surgeon or interventionalist to visualize the anatomy and shape of the vessel.

Conventionally, in order to introduce contrast material fluid into the vessel, the guide wire must be retracted from the catheter and contrast material fluid injected through the catheter. This prolongs the procedure, thus increasing the risk to the patient and may also cause loss of vascular access.

There is, therefore, a clear need for a catheter that allows to introduce contrast material fluid without the need to retract a guide wire.

### SUMMARY OF THE INVENTION

This invention pertains to a catheter that allows to introduce contrast material fluid without the need to retract a guide wire.

In the first embodiment of this invention, a catheter comprises an elongate body with a lumen extending between a distal end and a proximal end of the elongate body. The lumen has a round cross-section proportioned to simultaneously slidably receive a guide wire and to provide a path for a contrast material fluid. There is a one way valve disposed at the proximal end to prevent backflow of the contrast material fluid through the proximal end.

There is also a conduit tube comprising an inlet and an outlet for supplying the contrast material fluid. The conduit tube is attached to the elongate body of the catheter adjacent to its proximal end in the way that the outlet is in fluid communication with the lumen. This allows the contrast material fluid introduced into the inlet to be delivered to and flushed through the lumen.

The conduit tube further comprises a syringe adaptor for coupling a syringe containing the contrast material fluid and the conduit tube. The syringe adaptor is attached to the inlet. The syringe adaptor further comprises a flow valve for controlling the flow of the contrast material fluid from the syringe to the conduit tube.

In the second embodiment of this invention, a catheter comprises an elongate body with a first lumen extending between the distal end and the proximal end of the elongate body. The first lumen has a round cross-section proportioned to slidably receive a guide wire. There is also a second lumen extending between the distal end and the proximal end of the elongate body. The second lumen has no fluid communication with the first lumen and has a cross-section proportioned to provide a path for a contrast material fluid. The second lumen is sealably enclosed at the proximal end.

There is also a conduit tube comprising an inlet and an outlet for supplying the contrast material fluid. The conduit tube is attached to the elongate body of the catheter adjacent to its proximal end in the way that the outlet is in fluid communication with the second lumen. This allows the contrast material fluid introduced into the inlet to be delivered to and flushed through the second lumen.

The conduit tube further comprises a syringe adaptor for coupling a syringe containing the contrast material fluid and the conduit tube. The syringe adaptor is attached to the inlet. The syringe adaptor further comprises a flow valve for controlling the flow of the contrast material fluid from the syringe to the conduit tube.

### BRIEF DESCRIPTION OF THE DRAWINGS FIGURES

FIG. 1 depicts the catheter according to the first embodiment of this invention. FIG. 2 depicts a cross-section of the catheter according to the first embodiment of this invention. FIG. 3 depicts a partial cross-section of the catheter according to the second embodiment of this invention.

### DETAILED DESCRIPTION OF THE DRAWINGS FIGURES

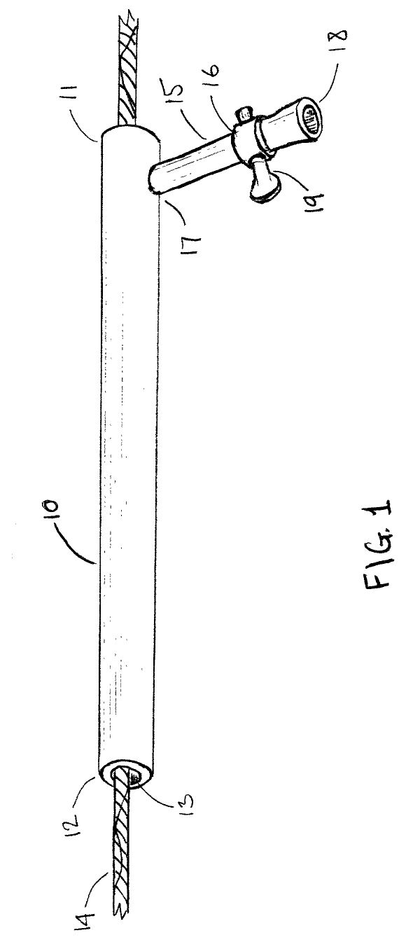

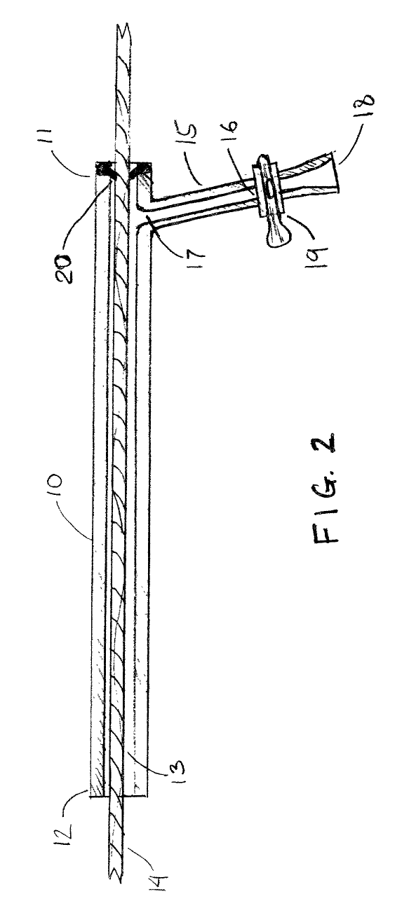

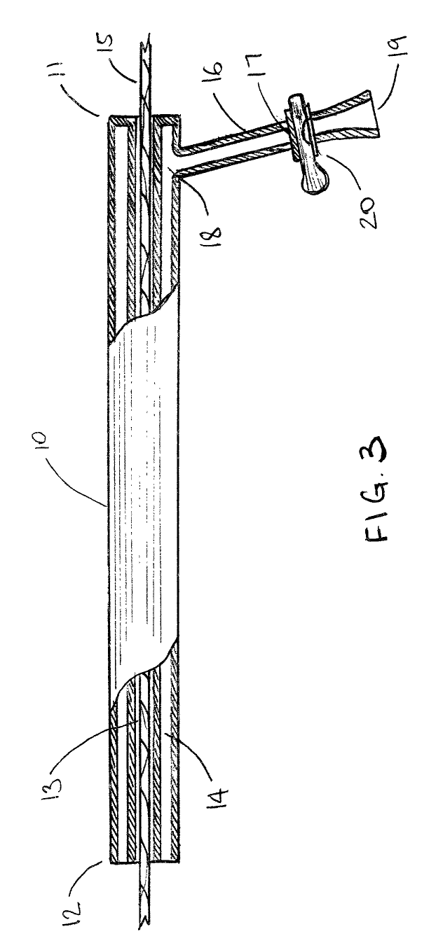

This invention will be better understood with the reference to the drawing figures FIG. 1, FIG. 2 and FIG. 3. The same numerals refer to the same elements in FIG. 1 and FIG. 2, but different elements in FIG. 3.

Viewing both FIG. 1 and FIG. 2, there is shown the catheter of the first embodiment of this invention (the cross-section thereof is depicted in FIG. 2). Numeral 10 indicates Elongate Body of the catheter. Numeral 11 indicated Distal End and numeral 12 indicates Proximal End of Elongate Body 10. Numeral 13 indicates Lumen. Lumen 13 extends between Proximal End 11 and Distal End 12. Numeral 14 indicates Guide Wire. Lumen 13 has a round cross-section proportioned to simultaneously slidably receive Guide Wire 14 and to provide a path for a contrast material fluid.

Numeral 15 indicates Conduit Tube. Conduit Tube 15 is used for supplying the contrast material fluid and comprises Inlet indicated by numeral 16 and Outlet indicated by numeral 17. The conduit tube is attached to Elongate Body 10 adjacent to Proximal End 11. Outlet 17 is in fluid communication with Lumen 13. This allows the contrast material fluid introduced into Inlet 16 to be delivered to and flushed through Lumen 13.

Numeral 18 indicates Syringe Adaptor. Syringe Adaptor 18 is used for coupling a syringe containing the contrast material fluid and Conduit Tube 15. Syringe Adaptor 18 is attached to Inlet 16.

Syringe Adaptor 18 further comprises Flow Valve indicated by numeral 19. Flow Valve 19 is used for controlling the flow or completely preventing the flow of the contrast material fluid from the syringe to Conduit Tube 15.

Numeral 20 indicates One Way Valve. One Way Valve 20 is disposed at Proximal End 11 in order to prevent backflow of contrast material fluid through Proximal End 11.

As can bee seen, the contrast material fluid can be introduced into the vessel by way of Lumen 13 without the need to retract Guide Wire 14.

Viewing now FIG. 3, there is shown the catheter of the second embodiment of this invention (including the partial cross-section thereof). Numeral 10 indicates Elongate Body of the catheter. Numeral 11 indicated Proximal End and numeral 12 indicates Distal End of Elongate Body 10. Numeral 13 indicates First Lumen. First Lumen 13 extends between Proximal End 11 and Distal End 12. Numeral 14 indicates Second Lumen. Second Lumen 14 extends between Proximal End 11 and Distal End 12. Second Lumen 14 has no fluid communication with First Lumen 13 and has a cross-section proportioned to provide a path for a contrast material fluid. Second Lumen 14 is sealably enclosed at Proximal End 11, which prevents the backflow of contrast material fluid through Proximal End 11.

Numeral 15 indicates Guide Wire. First Lumen 13 has a round cross-section proportioned to slidably receive Guide Wire 15.

Numeral 16 indicates Conduit Tube. Conduit Tube 16 is used for supplying the contrast material fluid and comprises Inlet indicated by numeral 17 and Outlet indicated by numeral 18. The conduit tube is attached to Elongate Body 10 adjacent to Proximal End 11. Outlet 18 is in fluid communication with Second Lumen 14. This allows the contrast material fluid introduced into Inlet 17 to be delivered to and flushed through Second Lumen 14.

Numeral 19 indicates Syringe Adaptor. Syringe Adaptor 19 is used for coupling a syringe containing the contrast material fluid and Conduit Tube 16. Syringe Adaptor 19 is attached to Inlet 17.

Syringe Adaptor 19 further comprises Flow Valve indicated by numeral 20. Flow Valve 20 is used for controlling the flow or completely preventing the flow of the contrast material fluid from the syringe to Conduit Tube 16.

As can bee seen, the contrast material fluid can be introduced into the vessel by way of Second Lumen 14 without the need to retract Guide Wire 15.

While the present invention has been described and defined by reference to the preferred embodiments of the invention, such reference does not imply a limitation on the invention, and no such limitation is to be inferred. The invention is capable of considerable modification, alteration, and equivalents in form and function, as will occur to those ordinarily skilled and knowledgeable in the pertinent arts. The depicted and described preferred embodiment of the invention is exemplary only, and is not exhaustive of the scope of the invention. Consequently, the invention is intended to be limited only by the spirit and scope of the appended claims, giving full cognizance to equivalents in all respects.

What is claimed is:

1. A catheter comprising:
(a) an elongate body having a proximal end and a distal end;
(b) a lumen extending between said distal end and said proximal end, said lumen having a round cross-section proportioned to simultaneously slidably receive a guide wire and to provide a path for a contrast material fluid;
(c) a one way valve disposed at the proximal end to prevent backflow of the contrast material fluid through the proximal end; and
(d) a conduit tube comprising an inlet and an outlet for supplying the contrast material fluid, said conduit tube attached to said elongate body adjacent to said proximal end in the way that said outlet is in fluid communication with said lumen so that the contrast material fluid introduced into said inlet can be delivered to and flushed through the lumen.

2. A catheter as in claim 1, wherein said conduit tube further comprises a syringe adaptor for coupling a syringe containing the contrast material fluid and said conduit tube, said syringe adaptor attached to said inlet.

3. A catheter as in claim 2, wherein said syringe adaptor further comprises a flow valve for controlling the flow of the contrast material fluid from the syringe to the conduit tube.

4. A catheter comprising:
(a) an elongate body having a proximal end and a distal end;
(b) a first lumen extending between said distal end and said proximal end, said first lumen having a round cross-section proportioned to slidably receive a guide wire;
(c) a second lumen extending between said distal end and said proximal end, said second lumen having no fluid communication with said first lumen and having a cross-section proportioned to provide a path for a contrast material fluid, said second lumen sealably enclosed at the proximate end; and
(d) a conduit tube comprising an inlet and an outlet for supplying the contrast material fluid, said conduit tube attached to said elongate body adjacent to said proximal end in the way that said outlet is in fluid communication with said second lumen so that the contrast material fluid introduced into said inlet can be delivered to and flushed through said second lumen.

5. A catheter as in claim 4, wherein said conduit tube further comprises a syringe adaptor for coupling a syringe containing the contrast material fluid and said conduit tube, said syringe adaptor attached to said inlet.

6. A catheter as in claim 5, wherein said syringe adaptor further comprises a flow valve for controlling the flow of the contrast material fluid from the syringe to the conduit tube.
